(19) United States
(12) Patent Application Publication
(10) Pub. No.: US 2010/0261747 A1
(43) Pub. Date: Oct. 14, 2010

(54) NOVEL PHARMACEUTICAL COMPOSITIONS AND METHODS OF PREPARATION AND USE

(76) Inventor: Peter D. Winch, New Albany, OH (US)

Correspondence Address: HOFFMANN & BARON, LLP, 6900 JERICHO TURNPIKE, SYOSSET, NY, 11791, US

(21) Appl. No.: 12/808,869

(22) PCT Filed: Dec. 17, 2008
(86) PCT No.: PCT/US08/87165

§ 371 Date: Jun. 17, 2010

Related U.S. Application Data
(60) Provisional application No. 61/008,016, filed on Dec. 18, 2007.

(51) Int. Cl. A61K 31/485; G01N 33/00; G01N 21/00; A61K 31/454; A61K 31/4468; A61K 31/4535

(52) U.S. Cl. 514/282; 436/92; 436/164; 514/326; 514/329

(57) ABSTRACT

The present invention provides a standardized method for the detection of narcotic abuse. Narcotics are formulated with a test compound that is not completely metabolized by the body. Narcotic diversion is detected by urinalysis of the suspected abuser, wherein the test compound may be detected even if the diverted narcotic is completely metabolized by the body, or otherwise undetectable in the urine.

### FIELD OF THE INVENTION

The present invention relates to pharmaceutic compositions, and in particularly to injectable narcotics compositions, their methods of manufacture, as well as their method of use. More particularly, the inventive compositions may be used to detect whether a narcotic drug was taken by a human.

### BACKGROUND OF RELATED TECHNOLOGY

Chemical dependency is a disease that afflicts healthcare professionals. A recent study cited roughly 1% of anesthesiologists in France suffering from addiction to opiates, (see Ann Fr Anesth Reanim. 2005 May; 24(5):471-9). Similar numbers have been cited for anesthesiology residents in the United States. Although numerous methods for detecting the abuse of narcotics have been described, these tests have proven inadequate in practice because of the short half-lives of the compounds, the weak circulating concentrations, and the tactics deployed by the abusers to evade detection (see Kintz P. Forensic Sci Int. 2005 Oct 4; 153(1):81-4 and Wang L. J Anal Toxicol. 2006 June; 30(5):335-41).

One of the most common ways to screen suspected diverters is analysis of their urine. Many analytic techniques have been developed for the determination of morphine in urine, including gas chromatography—mass spectrometry (see G. F. Grinsttead J. Anal. Toxicol. 15: 293-298, 1991; D. C. Fuller, W. H. Anderson J. Anal. Toxicol. 16: 315-318, 1992; and T. Vu-Duc, A. Vernay Biomed. Chromatogr. 4 : 65-69, 1990), immunoanalysis (see K. Aoki, Y. Shikama, A. Kokado, T. Yoshida, Y. Kuroiwa Forensic Sci. Int. 81: 125-132, 1996; and J. Beike, H. Kohler, B. Brinkmann, G. Blaschke J. Chromatogr. B 726: 111-119, 1999.), high performance liquid chromatography (see M. Pawula, D. A. Barrett, P. N. Shaw J. Pharm. Biomed. Anal. 11: 401-406, 1993; R. F. Venn, A. Michalkiewicz J. Chromatogr. Biomed. Appl. 525: 379-388, 1990; and B. A. Rashid, G. W. Aherne, M. F. Katmeh, P. Kwasowski, D. Stevenson J. Chromatogr. A 797: 245-250, 1998), and chemiluminescence (see R. W. Abbort, A. Townshend, R. Gill Analyst 111: 635-640, 1986; and Yunhua He, Jiuru Lu, Mei Liu, Jianxiu Du, Fei Nie J. of Anal. Toxicol. 29: 528-532, 2005). The present state of the art for the detection of narcotics includes multiple tests, multiple samples, multiple methods and multiple opportunities for the abuser to evade detection because of the limitations of the current techniques (see Wang L. J Anal Toxicol. “Analysis of 13 fentanils, including sufentanil and carfentanil, in human urine by liquid chromatography-atmospheric-pressure ionization-tandem mass spectrometry” 2006 June; 30(5):335-41).

The detection of certain narcotics, such as alfentanil, morphine, hydromorphine, fentanyl, sulftenanil, meperidine, remifentanil, among others,are particularly difficult and requires costly and sometimes inaccurate detection methods.

Addicted healthcare workers are a risk to society. These individuals predominantly divert substances of pharmaceutical grade that have been intended for the treatment of patients.

Based on the foregoing, a sensitive, fast, cost-efficient and standardized method for the detection of narcotic diversion and abuse is needed.

### SUMMARY OF THE INVENTION

In one aspect of the invention there is provided a composition, which includes a pharmaceutical agent, such as a drug, therapeutic or bioeffecting agent; and a dye suitable for use in the body, said dye being used in amounts sufficient to detect its presence or a metabolite of said dye in excreted urine. The pharmaceutical agent is desirably a narcotic agent, but may selected from any drug, therapeutic agent or bioeffecting agent useful for their intended purpose. Such compositions are particularly useful for detecting whether a human has been admistered the composition due to the easily detectable amounts of the dye excreted in the urine. Such tests may be particularly useful for detecting unlawful use or whether a patient has properly complied with their regimen.

In another aspect of the invention there is provided a standardized method for the detection of narcotic abuse. Narcotic compositions are prepared with the addition of an analyte, said analyte including at least one dye suitable for use in the human body, and that is not completely metabolized by the body. The dye is present in the composition in amounts which permits its detection in urine. Narcotic diversion may be detected by urinalysis of the suspected abuser, wherein the test compound may be detected even if the diverted narcotic is completely metabolized by the body, or otherwise undetectable in the urine.

In some embodiments of the invention, the dye is visually detectable in the urine of a human who has been administered the composition. In other embodiments of the invention, the dye is not visually detectable such that the results will not be readily obvious to the person being tested. The choice of detection method may vary, as will be discussed further herein, and will in part depend on the amount of dye added to the composition and the selected dosage form to be used.

In another aspect of the invention, there is provided a pharmaceutical composition, including:
a.) a narcotic substance; and
b.) an analyte which includes a dye suitable for use in the human body in an amount sufficient to detect its presence or a metabolite thereof once excreted in urine.

In another aspect of the invention, there is provided a method of making a pharmaceutical composition which provides a detectable non-narcotic-derived component in the urine of the user, the method including the step of:
a.) providing a composition which includes a narcotic substance
b.) mixing into said composition a dye suitable for use in the human body, said dye being present in an amount sufficient to detect its presence or a metabolite thereof subsequent to excretion in the urine.

In another aspect of the invention, there is provided a method of detecting the presence of a narcotic composition in urine comprising:
a.) providing a composition of a narcotic substance and a dye suitable for use in the human body; said dye being present in an amount sufficient for its detection or the detection of a metabolite thereof once excreted in the urine;
b.) obtaining a sample of urine from a suspected user of said composition; and
c.) detecting the presence of the dye or a dye metabolite in the urine as an indicator that the suspected user has taken the narcotic substance.

In another aspect of the invention, there is provided a method of monitoring narcotic diversion comprising the steps of:
adding a dye suitable for use in the body and in an amount sufficient for detection once excreted in urine to the formulation of an injectable narcotic; and
b.) testing the urine stream of a suspected diverter for the presence of the dye or a dye metabolite.

### DEFINITIONS

As used herein, the term “oral dosage form or composition” is intended to mean a composition of the present invention intended for oral ingestion, wherein said oral composition includes, but is not limited to solid tablets, powders, capsules, suspensions, lozenges, films, patches or lollipops.

As used herein, the term “alfentanil” is intended to mean N-[1-[2-(4-ethyl-5-oxo-tetrazol-1-yl)ethyl]-4-(methoxymethyl)-4-piperidyl]-N-phenyl-propanamide as shown in Formula 1, and its pharmaceutically acceptable salts.

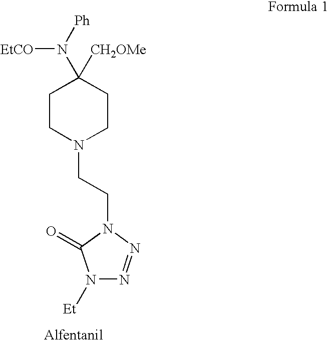

As used herein the term “hydromorphone” is intended to mean 4,5-epoxy-3-hydroxy-17-methylmorphinan-6-one, as shown in Formula 2, and its pharmaceutically acceptable salts.

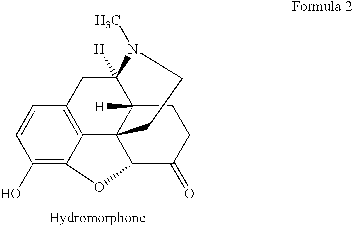

As used herein, the term “morphine” is intended to mean the compound shown in Formula 3, and its pharmaceutically acceptable salts.

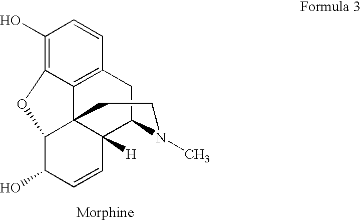

As used herein, the term “sufentanil” is intended to mean N-[(4-(Methoxymethyl-1-(2-(2-thienypethyl)-4-piperidinyl)]-N-phenylpropanamide, as shown in Formula 4, and its pharmaceutically acceptable salts.

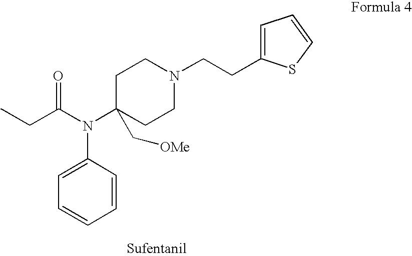

As used herein, the term “fentanyl” is intended to mean N-(1-phenethyl-4 piperidyl) propionanilide, as shown in Formula 5, and its pharmaceutically acceptable salts.

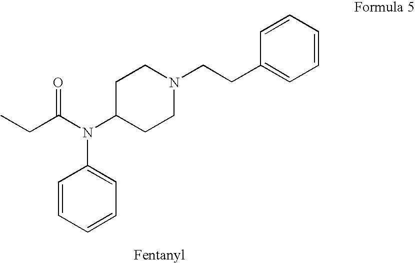

As used herein the term “meperidine” is intended to mean the compound shown in Formula 6, and its pharmaceutically acceptable salts.

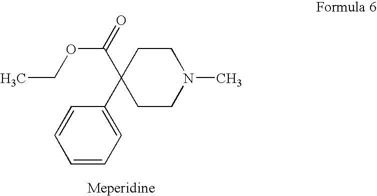

As used herein, the term “remifentanil” is intended to mean methyl 1-[2-methoxycarbonylethyl)-4-(phenyl-propanoyl-amino)-piperidine-4-carboxylate, as shown in Formula 7, and its pharmaceutically acceptable salts.

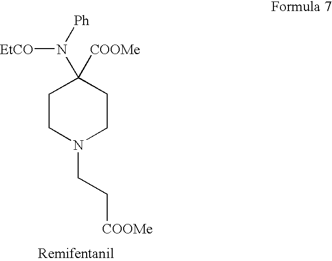

As used herein, the term “methylene blue” is intended to mean 3,7-bis(dimethylamino)phenazathionium chloride, as shown in Formula 8, and its pharmaceutically acceptable salts.

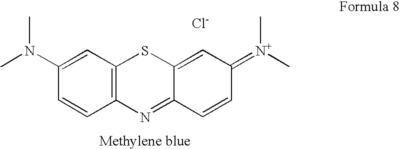

As used herein, the term “indigo carmine” is intended to mean 5,5′-indigodisulfonic acid disodium salt, as shown in Formula 9, and its pharmaceutically acceptable salts

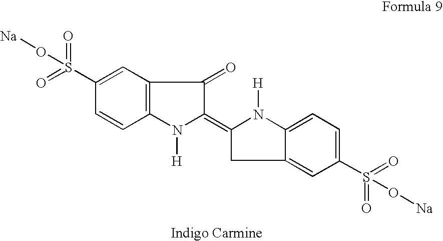

As used herein the term “narcotic” is intended to mean a drug which acts as an agonist at opioid receptors including the mu, and/or kappa and/or delta receptor subtypes. Narcotics include, by way of example only, but are not limited to the following: morphine, meperidine, hydromorphone, fentanyl, methadone, hydrocodone, oxycodone, sufentanil, remifentanil, morphine-6-gluconate, and alfentanil.

As used herein, the term “narcotic diversion” is intended to mean the inappropriate or unauthorized use of legally manufactured pharmaceutical narcotics.

As used herein the term “pharmaceutically acceptable anion” refers to any one of a group of inorganic or organic anions known in the art which balance the charge of the cationic drug. Examples of such anions include the following: chloride, bromide, sulfate, phosphate, methanesulfonate, formate, acetate, trifluoroacetate, citrate, fumarate, malate, tartate, succinate, and salicylate.

As used herein the term “pharmaceutically acceptable salt” refers to any one of a group of inorganic or organic acids known in the art which are combined with the free base form of a drug. Examples of such acids include, without limitation, the following: hydrochloric acid, sulfuric acid, phosphoric acid, methanesulfonic acid, formic acid, acetic acid, citric acid, fumaric acid, maleic acid, tartaric acid, succinic acid, and salicylic acid.

As used herein the term “urinalysis” is intended to mean analysis of the urine, wherein the analyte may be any of a number of compounds, including but not limited to dyes, such as methylene blue, and indigo carmine, and their metabolites, such as leucomethylene blue,and Azure B. Urinalysis is intended to mean the same thing as “analysis of the urine stream”.

### DETAILED DESCRIPTION OF THE INVENTION

The narcotics useful in the present invention include any narcotic suitable for use in mammals, and particularly for use in humans. These narcotics may be selected from a variety of compounds which may be derived from natural or synthetic sources. Of particular significance are those narcotics selected from the group consisting of alfentanil, morphine, hydromorphine, methadone, hydrocodone, oxycodone, fentanyl, suftentanil, meperidine, remifentanil and their pharmaceutically acceptable salts. These particular narcotics are widely used and are available in healthcare institutions and doctor's offices for administration to patients. These narcotic drugs are often administered in their pharmaceutically acceptable salt form for increased bioavailability and efficacy.

The narcotics may be present in the inventive compositions in various amounts, depending on the dose to be administered and the route of administration. For example, amounts of about 0.01 mg to about 1000 mg may be employed, as well as various amounts in between this range, such as 5, 10, 20 and 100 mg doses. If the composition is an injectable form, is is usually referred to as mg/mL. In such cases the amount of narcotic present may be from about 0.01 mg/mL to about 100 mg/mL, although these amounts may also vary depending on the chosen dosage regimen.

Dyes useful in the present compositions include any dyes which are suitable for use in the human body. Such dyes include, but are not limited to: FD&C blue #1, FD&C blue #2, methylene blue, indigo carmine, fluorescein, indocyanine green, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid. Preferred dyes are methylene blue, and indigo carmine.

The presence of the dye in the compositions is in an amount sufficient to allow for its detection or the detection of its metabolites in the urine of a patient or user of the composition. In other words, desirably, at least a portion of the dye is not metabolized and fully passes through the body and into the urine. In such cases, detection of the dye may be easily observable or detectable by simple test methods. In cases where the color of the dye is not readily observable or easily detectable in the urine, other more sophisticated methods may be employed, such as spectrophotometry. It is preferred that the detection method used for detecting the presence of dye or the dye metabolites in urine either be through observation of the color or through a color indicating test method; such as a test strip which changes color in the presence of the dye or a test composition which can be added to the urine and which also changes color in the presence of the dye.

The dye is intended to be present in an amount which is detectable in the excreted urine of the user of the composition. Generally, the dye is present in compositions of the present invention in amounts of about 1 to about 1000 mg per dose. The dosage forms may be injectable, oral, or transmucal or transdermal or other known forms.

The drugs may be incorporated into the various dosage forms along with appropriate pharmaceutical additives such as solvents, diluents, adjuvents excipients taste-masking agents, fillers and sweeteners. For example, sterile diluents or solvents such as water, saline solution, fixed oils, polyethylene glycols, glycerine, and alcohols, such as ethyl alcohol, or other solvents may be employed. Solutions are particularly useful when the dosage form is to be injectable. The chosen narcotic drug is then combined in the appropriate solvent, along with the pharmaceutical excipients of choice, if any, and the dye. This solution or admixture thus forms in an injectable composition and can be administered to a human.

Other pharmaceutical additives, such as benzyl alcohol, methyl parabin, anti-oxidants, such as for example, ascorbic acid or sodium bisulfate may also be employed. Chelating agents such as EDTA and buffers, such as acetates, citrates or phosphates may be useful in the inventive compositions. The compositions may also include agents for the adjustment of tonicity, such as sodium chloride or dextrose. The parenteral preparations may be enclosed in ampules, disposable syringes or multiple dose vials made of glass or plastic. The preferred diluent or carrier is water.

Although injectable compositions are preferred, the compositions of the present invention may be formulated in dosage forms which include solutions, powders dispersions, suspensions, gels or solid compositions, as well as films and patches for mucosal and/or transdermal delivery.

An embodiment of the invention is a method of using a dye that is excreted in the urine, which includes incorporating said dye in an injectable narcotic formulation.

An embodiment of the invention is a method of using a dye that is excreted in the urine, which includes incorporating said dye in an injectable narcotic formulation, wherein said dye may be methylene blue or indigo carmine.

An embodiment of the invention is a method of using a dye that is excreted in the urine, which includes incorporating said dye in an injectable narcotic formulation, wherein said dye may be methylene blue or indigo carmine, and wherein said narcotic is selected from alfentanil, morphine, hydromorphone, methadone, hydrocodone, oxycodone, fentanyl, sufentanil, meperidine, or remifentanil.

An embodiment of the invention is a method of using a dye that is excreted in the urine, which includes incorporating said dye in an injectable narcotic formulation, wherein said dye may be methylene blue or indigo carmine, and wherein said narcotic is selected from alfentanil, morphine, hydromorphone, methadone, hydrocodone, oxycodone, fentanyl, sufentanil, meperidine, or remifentanil. The dye may be present in an amount ranging from 1 to 1000 mg per injectable dose.

An embodiment of the invention is a method of using a dye that is excreted in the urine, which includes incorporating said dye in an oral narcotic formulation, wherein said dye may be methylene blue or indigo carmine, and wherein said narcotic is selected from alfentanil, morphine, hydromorphone, methadone, hydrocodone, oxycodone, fentanyl, sufentanil, meperidine, or remifentanil. The dye may be present in an amount ranging from 1 to 1000 mg per oral dose.

An embodiment of the invention includes a method for determining whether an individual has used a narcotic composition, said method including testing the urine stream of said individual for a non-narcotic dye-based analyte which was formulated with said narcotic.

An embodiment of the invention includes a method for determining whether an individual has used an oral, transdermal, transmucosal or injectable narcotic composition, said method including testing the urine stream of said individual for an analyte which is formulated with said injectable narcotic, wherein said analyte is a dye, and wherein said dye is methylene blue or indigo carmine, and wherein said narcotic is alfentanil, morphine, hydromorphone, fentanyl, sufentanil, meperidine, or remifentanil.

An embodiment of the invention includes a method for determining whether an individual has used an oral, transdermal, transmucosal or injectable narcotic composition, said method including testing the urine stream of said individual for an analyte which is formulated with said oral narcotic, wherein said analyte is a dye, and wherein said dye is methylene blue or indigo carmine, and wherein said narcotic is morphine, hydromorphone, methadone, hydrocodone, oxycodone, fentanyl, or meperidine.

In another embodiment of the invention, there is provided a method of monitoring narcotic diversion which includes the steps of:
1. Adding methylene blue to the formulation of an injectable narcotic.
2. Testing the urine stream of a suspected diverter for methylene blue.

In another embodiment of the invention there is provided a method of monitoring narcotic diversion which includes the steps of
1. Adding methylene blue to the formulation of an oral narcotic.
2. Testing the urine stream of a suspected diverter for methylene blue.

In another embodiment of the invention there is provided a method of monitoring narcotic diversion which includes the steps of:
1. Adding indigo carmine to the formulation of an injectable narcotic.
2. Testing the urine stream of a suspected diverter for indigo carmine.

In another embodiment of the invention there is provided a method of monitoring narcotic diversion which includes the steps of:
1. Adding indigo carmine to the formulation of an oral narcotic.
2. Testing the urine stream of a suspected diverter for indigo carmine.

In another embodiment, the present invention provides a method for empowering the legitimate purchaser of an injectable narcotic to test for its diversion, the method including manufacturing an injectable narcotic composition that contains a dye, wherein said dye is detectable in the urine stream of a-person into whom said injectable narcotic is subsequently injected. Any of the aforementioned narcotics, dyes and their relative amounts may be used.

In another embodiment, the present invention provides a method for empowering the legitimate purchaser of an oral narcotic to test for its diversion, said method including manufacturing a narcotic composition, said composition being in the form of an injectable, oral, transdermal or transmucosal dosage form, and which contains a dye, wherein said dye is detectable in the urine stream of a person to whom said composition was previously administered. Any of the aforementioned narcotics, dyes and their relative amounts may be used.

Another embodiment of the invention is a narcotic composition selected from the group consisting of:

A pharmaceutical solution which includes water, a pharmaceutically acceptable morphine salt in the range of 0.01 mg/mL to 100 mg/mL, and methylene blue in the range of 1 to 1000 μg/mL;

A pharmaceutical solution which includes water, a pharmaceutically acceptable alfentanil salt in the range of 10 μg/mL to 1500 μg/mL, and methylene blue in the range of 1 to 1000 μg/mL;

A pharmaceutical solution which includes water, a pharmaceutically acceptable hydromorphone salt in the range of 0.1 mg/mL to 2 mg/mL, and methylene blue in the range of 1 to 1000 μg/mL;

A pharmaceutical solution which includes water, a pharmaceutically acceptable sufentanil salt in the range of 1 μg/mL to 250 μg/mL, and methylene blue in the range of 1 to 1000 μg/mL;

A pharmaceutical solution which includes water, a pharmaceutically acceptable fentanyl salt in the range of 1 μg/mL to 250 μg/mL, and methylene blue in the range of 1 to 1000 μg/mL;

A pharmaceutical solution which includes water, a pharmaceutically acceptable meperidine salt in the range of 10 mg/mL to 500 mg/mL, and methylene blue in the range of 1 to 1000 μg/mL;

A pharmaceutical solution which includes water, a pharmaceutically acceptable morphine salt in the range of 0.01 mg/mL to 100 mg/mL, and methylene blue in the range of 1 to 20 μg/mL;

A pharmaceutical solution which includes water, a pharmaceutically acceptable morphine salt in the range of 0.01 mg/mL to 100 mg/mL, and indigo carmine in the range of 1 to 20 μg/mL;

A pharmaceutical solution which includes water, alfentanil hydrochloride in the range of 10 μg/mL to 1500 μg/mL, and methylene blue in the range of 1 to 20 μg/mL;

A pharmaceutical solution which includes water, alfentanil hydrochloride in the range of 10 μg/mL to 1500 μg/mL, and indigo carmine in the range of 1 to 20 μg/mL;

A pharmaceutical solution which includes water, hydromorphone hydrochloride in the range of 0.1 mg/mL to 2 mg/mL, and methylene blue in the range of 1 to 20 μg/mL;

A pharmaceutical solution which includes water, hydromorphone hydrochloride in the range of 0.1 mg/mL to 2 mg/mL, and indigo carmine in the range of 1 to 20 μg/mL;

A pharmaceutical solution which includes water, sufentanil citrate in the range of 1 μg/mL to 250 μg/mL, and methylene blue in the range of 1 to 20 μg/mL;

A pharmaceutical solution which includes water, sufentanil citrate in the range of 1 μg/mL to 250 μg/mL, and indigo carmine in the range of 1 to 20 μg/mL;

A pharmaceutical solution which includes water, fentanyl citrate in the range of 1 μg/mL to 250 μg/mL, and methylene blue in the range of 1 to 20 μg/mL;

A pharmaceutical solution which includes water, fentanyl citrate in the range of 1 μg/mL to 250 μg/mL, and indigo carmine in the range of 1 to 20 μg/mL;

A pharmaceutical solution which includes water, meperidine hydrochloride in the range of 10 mg/mL to 500 mg/mL, and methylene blue in the range of 1 to 20 μg/mL;

A pharmaceutical solution which includes water, meperidine hydrochloride in the range of 10 mg/mL to 500 mg/mL, and indigo carmine in the range of 1 to 20 μg/mL;

A particulate composition which includes remifentanil hydrochloride corresponding to remifentanil base in the range of 0.5 to 50 mg, and methylene blue in the range of 1 to 500 μg; and

A particulate composition which includes remifentanil hydrochloride corresponding to remifentanil base in the range of 0.5 to 50 mg, and indigo carmine in the range of 1 to 500 μg.

A pharmaceutical tablet, capsule, suspension, film, dispersion, patch, emulsion, lozenge, or lollipop incorporating methadone in the range of 1 mg/mL to 100 mg/mL, or 1 mg solid form to 100 mg solid form and methylene blue in the range of 1 to 200 mg.

A pharmaceutical tablet, capsule, suspension, film, dispersion, patch, emulsion, lozenge, or lollipop incorporating methadone in the range of 1 mg/mL to 100 mg/mL, or 1 mg solid form to 100 mg solid form and indigo carmine in the range of 1 to 200 mg.

A pharmaceutical tablet, capsule, suspension, film, dispersion, patch, emulsion, lozenge, or lollipop incorporating hydrocodone in the range of 1 mg/ml to 20 mg/ml, or 1 mg solid form to 20 mg solid form and methylene blue in the range of 1 to 200 mg.

A pharmaceutical tablet, capsule, suspension, film, dispersion, patch, emulsion, lozenge, or lollipop incorporating hydrocodone in the range of 1 mg/ml to 20 mg/ml, or 1 mg solid form to 20 mg solid form and indigo carmine in the range of 1 to 200 mg.

A pharmaceutical tablet, capsule, suspension, film, dispersion, patch, emulsion, lozenge, or lollipop incorporating oxycodone in the range of 1 mg/ml to 160 mg/ml, or 1 mg solid form to 160 mg solid form and methylene blue in the range of 1 to 200 mg.

A pharmaceutical tablet, capsule, suspension, film, dispersion, patch, emulsion, lozenge, or lollipop incorporating oxycodone in the range of 1 mg/ml to 160 mg/ml, or 1 mg solid form to 160 mg solid form and indigo carmine in the range of 1 to 200 mg.

In the above embodiments, methylene blue is intended to mean 3,7-bis(dimethylamino)phenazathionium chloride. Those of ordinary skill in the art will appreciate that other anions having the characteristics of chloride can be used without departing from the spirit and scope of the present invention.

In the above embodiments, indigo carmine is intended to mean the disodium salt of 5,5′-indigodisulfonic acid. Those of ordinary skill in the art will appreciate that other cations having the characteristics of sodium can be used without departing from the spirit and scope of the present invention.

Method of Use

Urinalysis may be performed by any of the methods known in the art (see A. R. DiSanto, J. G. Wagner J. Pharm. Sci. 61(7): 1086-1089, 1972; A. R. DiSanto, J. G. Wagner, J. Pharm. Sci. 61: 598-602, 1972; N. Belaz-David, L. A. Decosterd, M. Appenzeller, Y. A. Ruetsch, R. Chiolero, T. Buclin, J. Biollaz Eur. J. Pharm. Sci. 5(6): 335-345, 1997; J. Watanabe and R. Fujita. Chem. Pharm. Bull. 25(10): 2561-2567, 1977; Holger Borwitzky, Walter E. Haefeli, J″urgen Burhenne J. Chromatograt. B, 826: 244-251, 2005; and Norman F. Gaudette, Jon W. Lodge J. Anal. Toxicol. 29: 28-33, 2005, which are each incorporated by reference herein in their entireties).

Variations of these embodiments disclosed may become apparent to those of ordinary skill in the art upon reading the foregoing description. Accordingly, this invention includes all modifications and equivalents of the subject matter recited in the claims appended hereto. Moreover, any combination of the above-described elements in all possible variations thereof is encompassed by the invention unless otherwise indicated herein or otherwise clearly contradicted by context.

1. A pharmaceutical composition, comprising:
a.) a narcotic substance; and
b.) an analyte comprising a dye suitable for use in the human body in an amount sufficient to detect its presence or a metabolite thereof once excreted in urine.

2. The composition of claim 1, wherein the analyte is present in amounts of about 1 mg. to 1000 mg. per dose of the narcotic substance.

3. The composition of claim 1, wherein the dye is selected from the group consisting of methylene blue, indigo carmine and combinations thereof.

4. The composition of claim 1, wherein the narcotic substance comprises a compound selected from the group consisting of alfentenil, morphine, hydromorphine, fentanyl, sufentanil, meperidine, remifentanil, pharmaceutical salts and combinations thereof.

5. The composition of claim 1, further comprising at least one of a pharmaceutical exiginent and a solvent.

6. The composition of claim 1 in the form of an injectable fluid.

7. A method of making a pharmaceutical composition which provides a detectable non-narcotic-derived component in the urine of the user, the method including the step of:
a.) providing a composition which includes a narcotic substance
b.) mixing into said composition a dye suitable for use in the human body, said dye being present in an amount sufficient to detect its presence or a metabolite thereof subsequent to excretion in the urine.

8. The method of claim 7, wherein the dye is selected from the group consisting of methylene blue, indigo carmine and combinations thereof.

9. The method of claim 7, wherein the pharmaceutical composition further comprises a solvent for the narcotic substance.

10. The method of claim 7, wherein the pharmaceutical substance is in the form of an injectable fluid.

11. A method of detecting the presence of a narcotic composition in urine comprising:
a.) providing a composition of a narcotic substance and a dye suitable for use in the human body; said dye being present in an amount sufficient for its detection or the detection of a metabolite thereof once excreted in the urine;
b.) obtaining a sample of urine from a suspected user of said composition; and
c.) detecting the presence of the dye or a dye metabolite in the urine as an indicator that the suspected user has taken the narcotic substance.

12. The method of claim 11, wherein the detection is performed qualitatively.

13. The method of claim 11, wherein the detection is performed quantitatively.

14. The method of claim 12, wherein the detection is through visual observation.

15. The method of claim 13, wherein the detection is performed by a color indicator test.

16. The method of claim 13, wherein the detecting is performed by a spectrophotometer.

17. A method of monitoring narcotic diversion comprising the steps of:
a.) adding a dye suitable for use in the body and in an amount sufficient for detection once excreted in urine to the formulation of an injectable narcotic; and
b.) testing the urine stream of a suspected diverter for the presence of the dye or a dye metabolite.

18. The method of claim 17, wherein said narcotic is selected from the group consisting of alfentanil, morphine, hydromorphone, fentanyl, methadone, oxycodone, hydrocodone, sufentanil, meperidine, and remifentanil and their pharmaceutically acceptable salts.
